(19) United States
(12) Patent Application Publication
(10) Pub. No.: US 2026/0360485 A1
(43) Pub. Date: Nov. 12, 2026

(54) SYSTEMS AND METHODS TO ENHANCE HEALTHCARE COMMUNICATIONS

(71) Applicant: Rush University Medical Center, Chicago, IL (US)

(72) Inventors: Santosh Basapur, Aurora, IL (US); Jared Greenberg, Chicago, IL (US)

(21) Appl. No.: 19/102,061

(22) PCT Filed: Aug. 8, 2023
(86) PCT No.: PCT/US2023/071858

§ 371 Date: Feb. 7, 2025

Related U.S. Application Data
(60) Provisional application No. 63/395,968, filed on Aug. 8, 2022.

(51) Int. Cl. G16H 10/60; G16H 10/20; G16H 40/20; G16H 50/20

(52) U.S. Cl. CPC G16H 10/60; G16H 10/20; G16H 40/20; G16H 50/20

(57) ABSTRACT

A healthcare system that can include a plurality of input sources having a variety of different types of patient related information, including information pertaining to the condition, health, medical history and/or communication preferences, among other information, for each of a plurality of patients. The healthcare system can also include a communication content aggregator and curator system that can aggregate at least some of the patient information from at least some of the input sources, as well as information provided by a member(s) of a second group, and utilize the aggregated information in connection with customizing, including populating, a template containing that particular patient's information. The template can be used for, or to generate, communication content that is to be communicated to the member. Prior to being communicated to the member, the communication content be can be subjected to supervisory review, which can include amending the communication content.

### BACKGROUND

At times, a patient at a medical treatment facility, such as, for example, a patient at an intensive care unit (ICU) of a hospital, among other medical facilities, may not have the current capacity or ability to make decisions for themselves. In such situations, the patient may rely on surrogates, including one or more family members, among others, to make medical decisions for that patient. However, in at least certain situations, such surrogates may not have any advance or prior directive or guidance from the patient. Moreover, the surrogate may be unaware of the desires of the patient when the surrogate is to make potentially significant, if not life changing or altering, decisions on behalf of the patient. Thus, in such situations, the effect a patient's current medical status or condition may be having on the physical and/or mental well-being of the surrogate can be further exacerbated by the uncertainty and gravity of the decisions that the surrogate is to make on behalf of that patient. Under such circumstances, a surrogate can experience anxiety, depression, and/or stress, among other conditions, that can adversely affect the ability of the surrogate to comprehend information the surrogate is being provided by, and/or being asked to provide to, medical professionals. Such comprehension issues can also be further problematic based on the manner in which information is being communicated to the surrogate. For example, in such situations, under the strain and stress of the current situation, the surrogate may have difficulty comprehending and/or retaining verbal communications. Additionally, in such situations, verbal communications can have a tendency to be less accurate than other forms of communication.

ICU clinicians often aim to communicate with surrogates in ways that both inform the surrogates of a patient's medical condition and support the surrogate's decision-making and emotional needs. Ideally, such communications are provided with an aim of creating an inclusive environment for surrogates, provide scheduling for periodic meetings with the patient's family, and/or encourage the family presence in the ICU by patient bed-side. Yet, for a variety of reasons, such as, for example, time constraints and/or lack of appropriate training, among other reasons, clinicians sometimes experience difficulty implementing these recommended communication approaches. Similarly, surrogates may be unable, or not allowed, to be present in the ICU to meet with such clinicians as much as they would like, including for example, due to work obligations, personal commitments, outbreaks of diseases/illnesses, and/or guidelines or regulations of the associated medical facility.

### BRIEF SUMMARY

An aspect of the present disclosure is a healthcare system that can include a first database having an observation data pertaining to a patient, a second database having a historical medical data for the patient, and a third database having a customer preference data for the patient. The system can also include a communication content aggregator and curator system having at least one processor and a memory coupled with the at least one processor. The memory can include instructions that when executed by the at least one processor can cause the at least one processor to aggregate data at least obtained from the observation data, the historical medical data, and the customer preference data, and curate the aggregated data to provide a curated template. The curated template can be populated based at least in part on information provided by the aggregated data. Additionally, the memory can include instructions that when executed by the at least one processor can cause the at least one processor to generate a communication content comprising information from the curated template, and generate, in response to a receipt of a signal that indicates a supervisory approval of the communication content by one or more members of a first group, at least a signal that provides and/or indicates access to the communication content to at least one member of a second group.

Another aspect of the present disclosure is a method that can include accessing observation data pertaining to a patient, accessing historical medical data for the patient, and accessing customer preference data for the patient. The method can further include aggregating the observation data, the historical medical data, and the customer preference data to obtain a set of aggregated data, and curating the set of aggregated data to provide a curated template. The method can further include generating a communication content comprising information from the curated template, and generating, in response to a receipt of a signal that indicates a supervisory approval of the communication content by one or more members of a first group, at least a signal that provides and/or indicates access to the communication content to at least one member of a second group.

### BRIEF DESCRIPTION OF THE DRAWINGS

The description herein makes reference to the accompanying figures wherein like reference numerals refer to like parts throughout the several views.

FIG. 1 illustrates a healthcare communication system according to certain embodiments of the subject disclosure.

FIG. 2 illustrates an exemplary method for gathering and customizing at least portions of a template in connection with generating a summary from an interdisciplinary team of communication content for consideration for communication from a first group to one or more members of a second group.

FIG. 2A illustrates an exemplary template that can be utilized at least in connection with the formatting and selection of the communication content that is to be communicated to a second group.

FIG. 3 illustrates an exemplary demonstration of at least portions of a template being generated and customized to derive a summary of electrical medical records for consideration for communication from the first group to the one or more members of the second group.

FIG. 4 illustrates an exemplary demonstration of at least portions of a template being generated and customized to derive a summary of customer relationship management for consideration for communication from the first group to one or more members of the second group.

FIG. 5 illustrates an exemplary method of gathering communication content for review by one or members of the first group and that can be utilized for generating communication content for communication to a recipient device of one or more members of the second group.

The foregoing summary, as well as the following detailed description of certain embodiments of the present invention, will be better understood when read in conjunction with the appended drawings. For the purpose of illustrating the invention, there is shown in the drawings, certain embodiments. It should be understood, however, that the present invention is not limited to the arrangements and instrumentalities shown in the attached drawings.

### DESCRIPTION OF THE ILLUSTRATED EMBODIMENTS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

A number of features described below may be illustrated in the drawings in phantom. Depiction of certain features in phantom is intended to convey that those features may be hidden or present in one or more embodiments, while not necessarily present in other embodiments. Additionally, in the one or more embodiments in which those features may be present, illustration of the features in phantom is intended to convey that the features may have location(s) and/or position(s) different from the locations(s) and/or position(s) shown.

The subject disclosure provides a system that can automatically, or semi-automatically, enhance, support, and/or facilitate communications between a first party or group, such as, for example, care and service providers, among others, of a provider healthcare system, and a second group that can comprise one or more of the patient (e.g. the individual receiving medical services or care, collectively referred to herein as the care recipient), medical professionals, and/or surrogates of the care recipient (patient or customer) of the provider healthcare system, as well as any combinations thereof. The second group can also, according to certain embodiments, include other medical professionals that may also be assisting with the care management of the patient, including, for example, medical professionals that may be providing a second opinion or other support or care services, including, but not limited to nurses, among others. Moreover, the subject disclosure can be implemented for a wide range of patients, including, but not limited to ICU patients, inpatients, and outpatients. Additionally, the subject disclosure can be implemented during a variety of time periods, including, but not limited to, before, during, and/or after a prognosis, treatment, recovery, and/or rehabilitation stage(s) of a patient's treatment, as well as combinations thereof and time periods thereafter.

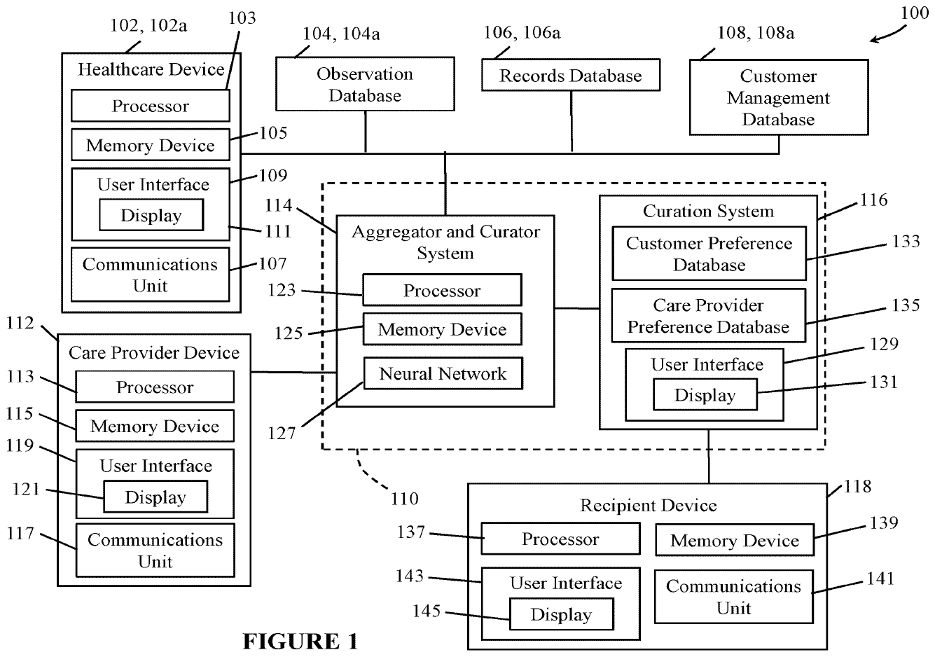

In a non-limiting illustration, certain patients can be incapacitated for a period of time, including, for example, be a patient that requires mechanical ventilation for a plurality of days. In such situations, at least one family member of the patient, among other members of the second group, can be identified as a decision maker for the patient, also referred to herein as a surrogate for that patient. Accordingly, the surrogate can provide consent to engage in communications with a healthcare communication system 100 (FIG. 1) of the subject disclosure, and, moreover, engage in communications in the form of written communications. Such consent can include the surrogate receiving at least periodic written communications from the system 100, such as, for example, daily or semi-daily written updates via email or hard copy, among other types of written or non-written communications. Such consent can also facilitate the surrogate providing written communications relating to patient information. Compared to verbal exchanges, particularly when considering the anxiety and emotional condition of the surrogate in view of the medical status of the patient, written communications, as generated by, or communicated to, the system 100 disclosed below having patient information can have a tendency to be more accurate and/or complete, and can assist in the comprehension and decision making on the part of the surrogate.

FIG. 1 illustrates a healthcare communication system 100 according to an illustrated embodiment of the subject disclosure. As seen in FIG. 1, the healthcare communication system 100 can utilize input from a plurality of input sources 102, 104, 106, 108, and can automatically generate and/or configure a first communication content to a communication content aggregator and curator system 110. Moreover, the input sources 102, 104, 106, 108 can provide a variety of different types of patient specific information that can be utilized by one or more individuals of a first group such as, for example, a clinician, care provider, or other agent of the healthcare system 100 in connection with generating communication content that is to be communicated to a surrogate of the second group. While for at least purposes of discussion, examples provided herein are discussed with respect to the communication of information to one or more surrogates of the second group. However, the embodiments disclosed herein are also applicable with respect to such communications being provided to other members of the second group, in addition to, or in lieu of, communications, if any, to a surrogate(s).

As seen in FIG. 1, a variety of different types of devices can be utilized for the input sources 102, 104, 106, 108. Moreover, the input sources 102, 104, 106, 108 can take a variety of forms, including, for example, comprise one or more databases, including, but not limited to, cloud based and/or non-cloud based databases, as well as combinations thereof. However, additional input sources 102 can also be implemented into the healthcare communication system 100 that are used in connection with an aggregation and curation of communication content relating to a subject patient.

For example, as seen in FIG. 1, according to certain embodiments, one example of an input source 102 can, for example, be one or more healthcare devices 102a having at least one processor 103 that can execute one or more instructions that may be stored on a memory device 105. Moreover, the memory device 105 can include instructions that, when executed by the processor 103, can cause the processor 103 to perform functions associated with the healthcare device 102a receiving, determining, measuring, recording, and/or detecting patient related information that the healthcare device 102a can communicate, via a communication unit 107, to one or more other devices or components of the healthcare communication system 100.

A variety of devices can be utilized as the healthcare device 102a, including, but not limited to, personal and/or mobile computing device(s), such as, for example, a desktop computer, laptop, tablet, smart phone, and/or mobile or cellular device, among other communication or computing devices, as well as combinations thereof. Additionally, or alternatively, according to certain embodiments, the healthcare devices 102a can comprise a medical device, including, but not limited to, diagnostic and/or monitoring device(s), that can be used to obtain, monitor, measure, and/or diagnose information regarding to a medical condition of the patient. Thus, according to certain embodiments, the one or more healthcare devices 102a can be, or include, a medical monitoring device that is operably coupled to a patient and which is utilized to detect, monitor and/or measure a medical or health status, level, and/or condition of the patient.

The healthcare device 102a, as well as other components of the system 100, can communicate within the system 100, and/or with external devices that may be communicatively connected to components of the system 100 in a variety of manners. For example, the communication unit 107 of the healthcare device 102a can be configured to communicate via internet enabled communications, a wireless connection (e.g., Wi-Fi, Bluetooth and, cellular, etc.), or a wireline connection (e.g., utilizes infrastructure delivering telecommunications to residences and or buildings), among other forms of communication.

The healthcare device 102a can also include a user interface 109 that can receive information from, and/or communicate information to, a user of the healthcare device 102a. For example, the user interface 109 can include a display 111, including, but not limited to a screen, monitor, or touch screen, among others. Additionally, or alternatively, the user interface 109 can include keyboard and/or mouse, as well as combinations thereof, among other input/output devices. Thus, for example, according to certain embodiments, the user interface 109, among other components of the healthcare device 102a, can be used to collect information from medical professionals, including, for example, information obtained during patient visits or check-ups. Thus, for example, the healthcare device 102a can be utilized to collect and/or record information, including, but not limited to, observations, from a member(s) of a care delivery team, including a physician(s), resident(s), and/or nurse(s), among others, that may be attained from scheduled or non-scheduled visits to the patient, including, while conducting rounds of patient visits. According to certain embodiments, the healthcare device 102a can be utilized to generate and/or store written information, scanned information, and audio files, as well as transcribe such audio files so as to provide a written transcript of such audio files.

According to certain embodiments, another input source 104 for the healthcare communication system 100 can be an observation database 104a that retains records or information indicative of information collected by, and/or observations of, members of the first group, among other sources of information. Such information can include, but is not limited to, patient observations and data, including clinical data captured during medical rounds by physicians and other experts of the first group. Additionally, according to certain embodiments, the observation database 104a can be a database that can automatically or semi-automatically receive and/or retrieve, as well as store, information stored by, or otherwise generated using, the healthcare device 102a. Additionally, or alternatively, the observation database 104a can be a database that can automatically or semi-automatically receive and/or retrieve, as well as store, information provided by other medical or monitoring devices, including, but not limited to, clinical information from equipment, such as, for example, monitoring devices that may be monitoring and/or measuring physical characteristics and/or status information of a patient and/or from associated specimens or samples from the patient. For example, according to certain embodiments, the observation database 104a can store information such as, for example, blood pressure levels, heart rates, oxygen levels, neurological functions, and/or body temperature(s), among other types of monitored or measured information.

According to certain embodiments, another input source 106 for the system 100 can be a records database 106a, such as, for example, a medical records database and/or system. For example, according to certain embodiments, the records database can be records or information from one or more electronic medical records (EMR) systems that may be associated with one or more related and/or unrelated healthcare providers. Thus, the records database 106a can include, or provide information from, patients' medical records, as well as related information. Such records can contain, for example, historical medical information for one or more patients, as well as other background information, including information regarding medical procedures the patient has undergone, medications or prescriptions for the patient, family medical histories, backgrounds, ethnicity, and/or sex, among other patient related background information.

Another exemplary input source 108 for the system 100 can be a customer management database 108a that can maintain information regarding the preferences relating to communications between at least the first group and the second group, such as, for example to a patient and/or a surrogate of the second group, including, for example, one or more family members of the patient, among other customers or customer representatives. According to certain embodiments, the customer management database 108a can include information from a customer relationship management system of the associated provider healthcare system or similar information of a provider healthcare system(s). For example, the customer management database 108a can provide contact information relating the surrogate(s) of the second group 108 that is/are, and/or is/are not, to be contacted by the first group or system 100 when providing patient information to the second group. In addition to providing contact information, the customer management database 108a can also indicate communication preferences for the patient or surrogate of the second group, including, for example, preferences in the manner in which information is communicated from the first group or the system 100. Such preferences can include, for example, an indication of whether communications from the first group or system 100 are to be provided to the second group via traditional mail, electronic mail (email), text messages, postings in software applications, and/or telephonic calls or messages, among other manners of communication.

Information can be exchanged between the input sources 102, 104, 106, 108, such as, for example, the above-discussed healthcare device 102a, observation database 104a, records database 106a, and/or customer management database 108a, as well as various combinations thereof, and a communication content aggregator and curator system 110 via any suitable network of the system 100, including, but not limited to, via a local area network (LAN), private network internet, Ethernet, storage area network, and/or wide area network (WAN), among other types of networks of forms or protocols for wired and/or wireless communications. Additionally, information can be exchanged between the communication content aggregator and curator system 110 and one or more client or care provider devices 112 that can be utilized by one or more members of the first group. Such care provider devices 112 can include, but are not limited to, mobile phones, cellular phones, smart devices, tablets, personal computers, and/or laptops, among other computing devices. Thus, according to certain embodiments, the care provider devices 112 can include at least one or more processors 113, a memory device 115, a communication unit 117, and/or a user interface 119, including a display 121, that is similar to the processor 103, memory device 105, communication unit 107, user interface 109, and display 111 discussed above with respect to the healthcare device 102a. Thus, in at least some situations, such communications between the communication content aggregator and curator system 110 and a care provider device 112 can be sent via a wired and/or wireless communication, and thus can be sent and/or received in the form of a text message, email, and/or automated call, among other forms of messaging and communication. Further, according to certain embodiments, the communication content aggregator and curator system 110 and one or more of the care provider devices 112 may, or may not, be separate devices or systems, or, alternatively, be part of the same device or system.

The communication content aggregator and curator system 110, including, for example, an aggregator and curator system 114 of the communication content aggregator and curator system 110, can be a computing device that includes one or more processors 123, and can further include, or be communicatively coupled to, a memory device 125. Moreover, similar to other devices discussed herein having a processor and memory device, the processor 123 can execute one or more instructions that may be stored on the associated memory device 105. Further, according to certain embodiments, the communication content aggregator and curator system 110 can include, or be communicatively coupled to, a neural network 127. The neural network 127 can apply data and information to one or more neural network algorithms, such as but not limited to a multilayer perceptron (MLP), a restricted Boltzmann Machine (RBM), a convolution neural network (CNN), and/or any other neural network algorithm that will be apparent to those skilled in the relevant art(s) without departing from the spirit and scope of the disclosure.

As seen in FIG. 1, according to certain embodiments, the aggregator and curator system 114 and/or the communication content aggregator and curator system 110 can automatically obtain information from one or more of the input sources 102, 104, 106, 108, and/or from one or more members of the first group the care provider device 112. Such information can be, but is not limited to, a summary of a portion of the information derived, recorded, reported, and/or maintained in, and/or via use of, the one or more of the input sources 102, 104, 106, 108, and can be collected in the aggregate. As discussed below, the collected information can be utilized to generate communication content that is reviewable and/or editable by one or members of the first group before being transmitted, or otherwise communicated, to the surrogate. The communication content aggregator and curator system 110 can present, via, for example, communication(s) from the aggregator and curator system 114 to the care provider device 112, an aggregation of collected information, including, but not limited to, in an organized or prearranged format(s), for the communication content to the first group in a variety of different manners. For example, the information contained in the communication content can be arranged in a time ordered manner or in format provided by a template, among other formats or arrangements. Further, prior to, or following, aggregation of information from one or more of the input sources 102, 104, 106, 108, the member of the first group can select, such as, for example, via selections made using the user interface 119 of the care provider device 112, the information to be presented by the aggregator and curator system 114. Such selections by the member of the first group can be in a manner that allows that member to select which information is, and/or is not, to be included, or to be considered for inclusion, in the communication content that is to be received by the second group.

As mentioned above, according to certain embodiments, the communication content aggregator and curator system 110 can be adapted to provide a template in which information from one or more of the input sources 102, 104, 106, 108 can be selected for at least possible inclusion in the communication content. Thus, such a template can aid in the manual and/or automatic inputting of at least selected information into the template. According to such an embodiment, such use of a template, and associated information selected for the communication content, can accommodate such information for communication being conveniently formatted and/or presented in a generally consistent manner. As discussed below, in addition to such selections being used to customize the template with at least respect to specific types of information that is, or may be, communicated, the template can also be customized based on preferences and/or past experiences with the particular surrogate or second group that is to receive the communication.

In some embodiments, the neural network 127 of the communication content aggregator and curator system 110 can be configured to select, from the aggregated information, the particular information that is, and/or is not, to be provided in with the communication content that is delivered or transmitted to the second group. For example, according to certain embodiments, the neural network 127 can recognize one or more patterns with respect to the particular type of information that has, or has not, been provided to second groups, including, for example, with respect to selections historically made by members of the first group for patients having particular medical conditions, statuses, projections, historical information and/or preferences, among other criteria. Using at least such pattern recognition, the neural network 127 can, for example, determine, including predict and/or identify, the information that is, or is not to be communicated to a second group associated with particular patients. Additionally, the neural network 127 can also use machine learning to adjust such patterns based on patient or surrogate preferences and/or communications received by surrogates, including questions from surrogates and/or evaluations of the accuracies of information previously provided by the surrogates. The neural network 127 can also include a feedback loop in which information in communication content that is eventually, or has been, communicated to second groups, such as, for example, based on selections, reviews, or edits, by members of the first group, can be compared to determinations made by the neural network 127. The outcomes of such comparisons can be utilized by the neural network 127 to modify the pattern(s) or associated algorithm(s) utilized by the neural network 127 in connection with the neural network 127 determining which information is, or is not, to be included in communication content to second groups.

Additionally, or alternatively, the neural network 127 can utilize one or more patterns or algorithms to identify or develop a template providing the types, categories, or specific information that is to be included in communication content based on certain patient information. For example, the neural network 127 can utilize historical information regarding the categories and/or types of communication content that has been, or is, disclosed for patients having a variety of different medical conditions, statuses, characteristics, backgrounds, medical histories, and/or prognoses, among other information, and identify one or more patterns associated with the selection(s) of which information to include in a customized communication content for a particular patient and the associated patient's conditions/status. Moreover, such information, or derived patterns, can be utilized to develop one or more algorithms that can be used to identify a template indicating the type of information the first group, or member(s) thereof, is to disclose with communication content for a specific patient based on specific medical information for that patient. In determining such a customize template for a patient, the neural network 127 can also consider a variety of other information that may, or may not, be patient specific, including patient preferences, as indicated by the customer management database 108a and/or the customer preference database 133, and/or the care provider preference database 135. The neural network 127 can also include a feedback loop in which communication content that is eventually, or has been, communicated to second groups, such as, for example, based on amendments or changes by members of the first group to the templates derived by the neural network 127 can be compared to the template determinations made by the neural network 127. The outcomes of such comparisons can be utilized by the neural network 127 to modify the pattern(s) or associated algorithm(s) utilized by the neural network 127 in connection with the neural network 127 developing other templates.

According to certain embodiments, regardless of whether communication content for communication to the second group is manually selected by a member of the first group, and/or is automatically gathered, the healthcare communication system 100 and/or the communication content aggregator and curator system 110 can be configured such that communication content is not communicated to the surrogate without supervisory approval. For example, communication content be subjected to review and approval by a member of the first group, such as, but not limited to, medical staff of the associated provider healthcare system, among other possible individuals, employees, and/or agents. Such supervisory review can provide an opportunity of a member of the first group to not only review the communication content before the communication content is transmitted or otherwise communicated to the second group, but also edit such communication content, including, for example, removing, editing and/or supplementing the communication content.

Following supervisory approval of the communication content by a member of the first group, the approved communication content can be communicated from the aggregator and curator system 114 to a curation system 116 of the communication content aggregator and curator system 110. According to certain embodiments, the curation system 116 can be a database that stores and/or records information communicated to the curation system 116 from the communication content aggregator and curator system 110. Alternatively, or additionally, the curation system 116 can be a computing device having at least one or more processors and one or more memory devices, and which is accessible, or transmits communication content, to one or more members of the second group. For example, according to certain embodiments, the curation system 116 can include a user interface 129, including a display 131, from which the surrogate can receive the communication content from the communication content aggregator and curator system 110. Additionally, or alternatively, the curation system 116 can be utilized to communicate, including send, the communication content to one or more recipient devices 118 of a member of the second group, such as, for example, a surrogate, among other members of the second group.

The curation system 116 can take a variety of forms so as to accommodate the different communication preferences of the particular member(s) of the surrogate that is/are to receive the communication content from the first group. Further, as seen in FIG. 1, according to certain embodiments, the curation system 116 can be communicatively coupled to the recipient device 118. However, while the curation system 116 and the recipient device 118 are illustrated in FIG. 1 as separate components, according to other embodiments, the curation system 116, or a portion thereof, and the recipient device 118 can be part of the same component or system.

The ability of a surrogate to receive and/or access communication content, or the type of information that the surrogate is allowed to access in the via communication content provided by the curation system 116 or other portions of the communication content aggregator and curator system 110 can be limited. For example, the type of information contained communication content that a surrogate may receive can be limited, or at least partially controlled, by preferences that may have been established by the patient. Such patient, or customer, preferences may be recorded or otherwise stored at least by the customer management database 108a, and can be communicated to a customer preference sub-system or database 133 of the curation system 116. Additionally, or alternatively, the curation system 116 can store or record preferences of certain members, if not all, of the first group relating to the communication of the communication content to surrogates. For example, communication preferences, including preferences for the manner in which communications from the first group are made to the second group(s), and/or manners in which communications are made to the first group (or particular members of the first group), among other preferences of the first group, or its members, can, for example, be stored in a care provider preference database 135 of the curation system 116.

As previously mentioned, the curation system 116 can include the user interface 129, including the display 131, among other input/output (I/O) devices, that can be used to display commutation content provided by the curation system 116. Additionally, or alternatively, the user interface 129 can be utilized to provide, including patient information or certain preferences, by members of the first group and/or the surrogate or other members of the second group to the curation system 116 and/or to the communication content aggregator and curator system 110. Such a user interface 129 can also be utilized to provide preferences for members of the first group and/or the second group that may be stored in the associated customer preference database 133 or the care provider preference database 135.

A variety of different types of devices, including communication and/or computing devices, can be utilized as the recipient device 118. For example, according to certain embodiments, the recipient device 118 can include, but is not limited to, mobile phones, cellular phones, smart devices, tablets, personal computing devices, and/or laptops, among other personal computing devices. Thus, similar to the healthcare device 102a and/or the care provider device 112, the recipient device 118 can similarly include one or more processors 137 that is/are coupled to a memory device 139, a communication unit 141, and/or a user interface 143, including, for example, a display 145, among other input/output devices. Further, according to certain embodiments, the recipient device 118 can be configured to not only display and/or announce communication content from one or more communications communicated from the curation system 116, but can also include an input/output devices, such as, a keypad, touch screen, mouse, and/or microphone, among other input/output devices.

Accordingly, the recipient device 118 can accommodate input to the healthcare communication system 100 of information from the surrogate(s) of the second group. Such information, including feedback can, among other information, provide information, including responses, regarding the patient. For example, via use of the recipient device 118, a surrogate can provide information to the healthcare communication system 100 regarding patient observations by the surrogate or other members of the second group, and/or measured/recorded information that the surrogate or other members of the second group have made or obtained regarding the patient. In some situations, such information may pertain to observations by surrogate, or measurements taken of/from the patient, while the patient was at home or at another location or facility. Additionally, the recipient device 118 can also be utilized for purposes of scheduling appointments, and provide a forum for questions and/or feedback for a member(s) of the first group, among other communications. As seen in FIG. 1, such communications from the recipient device 118 can be received by the curation system 116 and sent from the curation system 116 to the aggregator and curator system 114. Additionally, such information communicated by use of the recipient device 118 can also be directly or indirectly communicated to the care provider device 110 of the first group, including to a care provider device 112, and/or provided, retrieved and/or recorded to/by one or more of the input sources 102, 104. 106, 108.

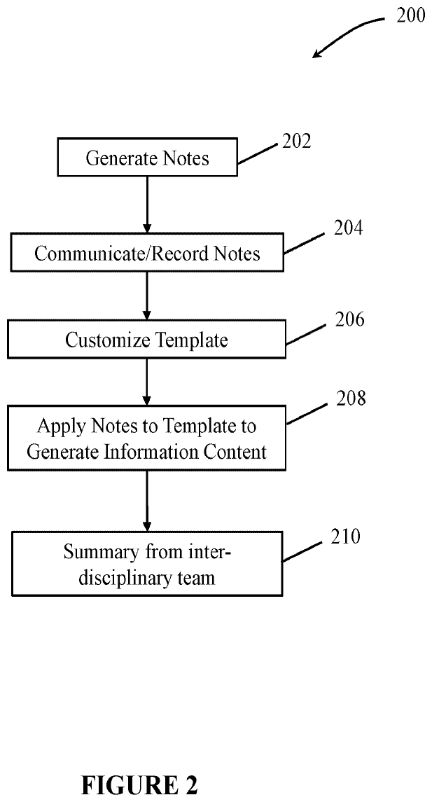

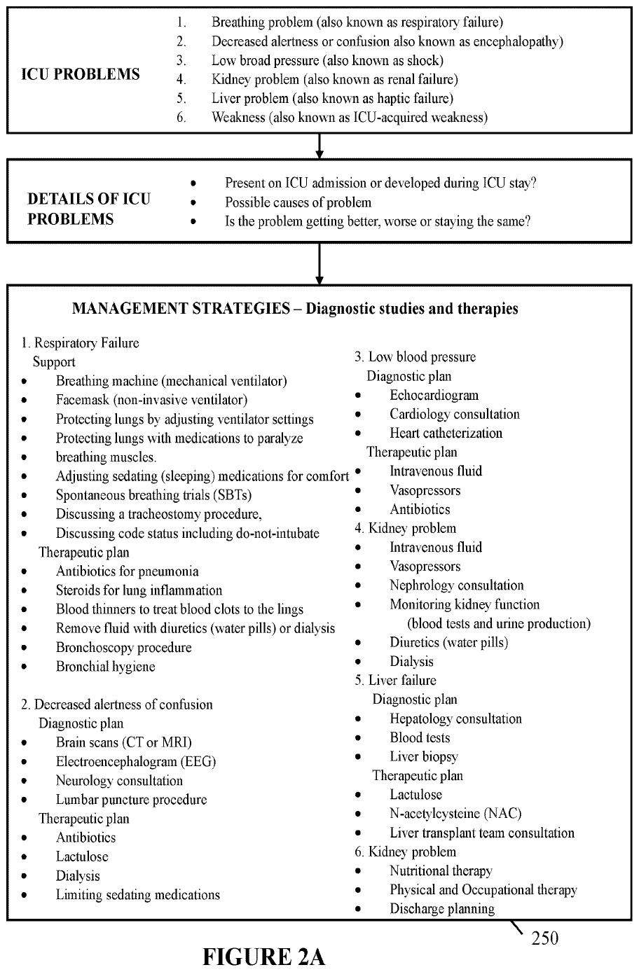

FIG. 2 illustrates an exemplary method 200 for gathering and customizing at least portions of a template in connection with generating a summary from an interdisciplinary team of communication content for consideration for communication from the first group to one or more surrogates of a second group. The method 200 is described below in the context of being carried out by the illustrated exemplary healthcare communication system 100. However, it should be appreciated that method 200 can likewise be carried out by any of the other described implementations, as well as variations thereof. Further, the method 200 corresponds to, or is otherwise associated with, performance of the blocks described below in the illustrative sequence of FIG. 2. It should be appreciated, however, that the method 200 can be performed in one or more sequences different from the illustrative sequence. Additionally, one or more of the blocks mentioned below may not be performed, and the method 200 can include steps or processes other than those discussed below.

At block 202, information can be obtained, generated, stored, and/or recorded regarding the patient. For example, according to certain embodiments, and/or in certain situations, information can be proved to, and stored by the observation database 104a. According to certain embodiments, at least a portion of the information provided to the observation database 104a can be information that is, or was, gathered, collected, or obtained by, or via use of, the healthcare device 102a. For example, the healthcare device 102a can be utilized by a member of the first group, including a medical professional, among others, such as, for example, to generate notes while performing, or in response to, visits to the patient, which can also be referred to as medical rounds. Such notes can be taken in a variety of manners, including, for example, via typing using the user interface 109 of the healthcare device 102a, or via use of a camera feature of the healthcare device 102a to scan handwritten notes. Additionally, or alternatively, such notes or information can be audio recorded by, or provided to the healthcare device 102a. Additionally, or alternatively, the healthcare device 102a be configured to transcribe such audio recordings, and/or audio detected by a microphone of the healthcare device 102a. Thus, for example, the healthcare device 102a can be configured to transcribe audio obtained from discussions or meetings involving one or members or teams of medical professionals from the first group. According to certain embodiments, at least a portion of such information obtained by, or via use of, the healthcare device 102a can be recorded or stored at the observation database 104a. The information generated at block 202 can also include information stored in one or more electronic medical records for a patient, including, but not limited to, clinical and non-clinical data that can be stored in at least the records database 106a.

At block 204, at least a portion of the information generated by the healthcare device 102a, the observation database 104a, and/or the records database 106a, can be communicated to, including received and/or retrieved by, the communication content aggregator and curator system 110, including, for example, an aggregator and curator system 114. Again, such communication of information to the communication content aggregator and curator system 110 can be attained in a variety of manners, including, but not limited to, via any suitable network.

At block 206, a template can be generated or retrieved that can be utilized in determining which of the information from block 204 may be used for communication content that is to be communicated by the system 100 to at least the recipient device 118. According to certain embodiments, the decision as to which information may be included in the communication content can be based, at least in part, on use of a template. Moreover, such a template can provide guidance as to the type of communication content that is to communicate, or considered for communication, to the second group. Such a template can also be used to provide consistency with respect the format of the communication content, including with respect to the consistency the curation system 116 communicates such information.

As previously discussed, according to certain embodiments, the template can be generate by a neural network 127. Alternatively, or additionally, the template can be derived using a variety of different criteria. For instance, in the example shown in FIG. 2, information being collected and/or retrieved relates to at least observations, discussions, and/or meetings of medical professionals of the first group that is stored the observations database 104a. Accordingly, in this example, at least a portion of a template can be generated that includes criteria relating to the type of information at issue, in this case information relating to, for example, medical observations. According to such an example, at least a corresponding portion of the template, or first portion of the template, can include criteria that is based, at least in part on the prevalence, or top, of potential medical problems that may be observed or detected by medical professionals of the first group.

For example, FIG. 2A illustrates a non-limiting example of at least such a portion of a template 250 that can also be utilized in connection with information provided by observations made, including recorded, by medical professionals of the first group. In the illustrated example, the illustrated template 250, or portion thereof, can be organized on the basis of an identified or observed ICU problem (e.g., “ICU PROBLEMS”), development, potential sources, and progress of the identified problem(s) (e.g., “DETAILS OF THE PROBLEM”), and various strategies and therapies for the identified problem(s) (e.g., “MANAGEMENT STRATEGIES—Diagnostic studies and therapies”), including, but not limited to, strategies regarding therapeutic and diagnostic plans, among other forms of treatment support.

For example, the exemplary template 250 shown in FIG. 2A illustrates six potentially prevalent ICU problems that can be commonly observed or identified among medical ICU patients, namely: (1) a breathing problem (respiratory failure), (2) decreased alertness or confusion (encephalopathy), (3) low blood pressure (shock), (4) a kidney problem (renal failure), (5) a liver problem (liver failure), and (6) ICU-acquired weakness. The template 250 also provides an indication of certain types of information to be provided relating to such problems, including if the problem was present before, or developed after, admission to the ICU, possible causes of the problem, and whether problem is getting better, worse, or staying the same. Additionally, for each of the six identified problems, the template 250A further includes an identification of the therapies or support to be taken for each problem, which can also be used to provide a template with respect to the type of information that is to be obtained for communications from the system 100 reporting on the status or condition of the patient to the surrogate. For example, with respect to problems related to liver failure, the template 250 can provide an indication that the communication to the surrogate can include results from associated blood test and a liver biopsy, as well as information from, or referral for, consultation with a hepatology specialist.

While the foregoing is discussed in terms of the template being used in connection with observations from the observation database 104a, and/or the healthcare device 102a, at least a portion of the information provided by the template 250 can be communicated to the surrogate, including in the form of a questionnaire. For example, according to certain embodiments, portions of the exemplary template 250 relating to six identified prevalent ICU problems, as well as portions of the template inquiring about details of the problem, can be communicated via the curation system 116 to the recipient device 118. The surrogate may then provide information or responses to the communicated portions of the template 250 that may be recorded, for example, in the observation database 104a, or otherwise evaluated in connection with the observations made by the medical professional of the first group. Thus, according to certain embodiments, the information provided at block 204 can further include information or response provided by the surrogate.

At block 208, at least a portion of the information provided at block 204 can be utilized in connection with customizing at least a portion of the template generated or retrieved at block 206. For example, using at least the information communicated at block 204, the template from block 206 can be customized at block 208, such as, for example, with respect to which information is, or is not, to be included in the template 250. Thus, for example, the categories or types of information that may, or may not, be relevant to the patient, and/or the current status or condition of the patient, as indicated by the information communicated at block 204, can be used to identify which portions of the template 250, and associated patient information, is, or is not, relevant to the patient. Accordingly, the template 250 can be configured so as to be used to determine which information is, or is not, to be populated. For example, if a patient is identified as having a particular problem(s), the template 250 can be configured to utilize information provided by block 204 to populate information relating to that problem(s), or categories associated with that problem(s) for consideration for inclusion in communication content for the surrogate. Further, the template 250 can also be configured to populate in the communication content additional information relating to that problem(s) or category, including, for example, instructions, explanations, and/or answers to anticipated questions, among other information relating to that problem(s).

With such an approach, the template 250, or portions thereof, can be customized to the patient. Moreover, such customization can assist in determining which information provided at block 204 is, or is not, to be at least considered for inclusion in the communication content for the surrogate. According to certain embodiments, the customization of the template 250 can further involve manual selections or identifications, such as, for example, selection by a member of the first group of a problem(s) being experienced by the patient and/or associated information based at least in part on the information provided at block 204 and through use of the user interface 119, 129 of the care provider device 112 and/or the curation system 116. Additionally, or alternatively, such a customization of the template 250 can be automatic, such as, for example, via the processor 123 of the aggregator and curator system 114 being operated in accordance with instructions from the memory device 125 and based, at least in part, on the type of information provided at block 204. Further, as previously discussed, such customization of the template 250 can be automatically done via use of the neural network 127.

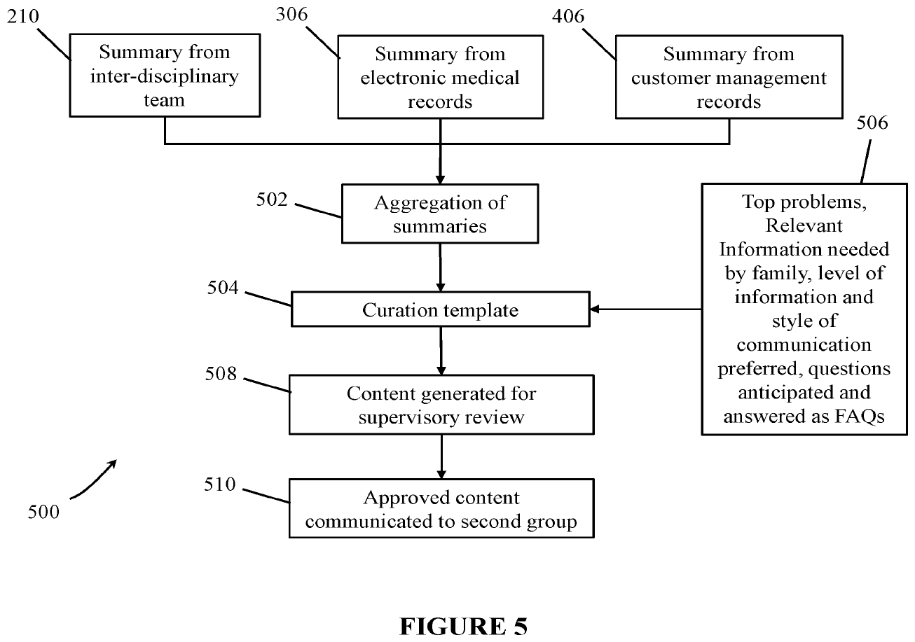

At block 208, at least a portion of the information provided to the communication content aggregator and curator system 110 at block 204, including, for example, the aggregator and curator system 114, can be applied to, or otherwise utilized in connection at least the portion of the template that was customized at block 206. The application of information from block 204 to the customized template can, at block 210, allow for the generation of a summary from inter-disciplinary review team. According to certain embodiments, the generated summary from inter-disciplinary review team can provide information that can be aggregated with other information for at least consideration for communication to the surrogate. Additionally, according to certain embodiments, the generated summary from inter-disciplinary review team can be formatted in a manner that can provide a portion, such as a first portion, of a template having patient specific information that is provided with the communication content. However, as discussed below with respect to FIG. 5, the generated summary from inter-disciplinary review team can also be subjected to review by one or more members of the first group before information contained in the generated summary from inter-disciplinary review team is communicated to the surrogate.

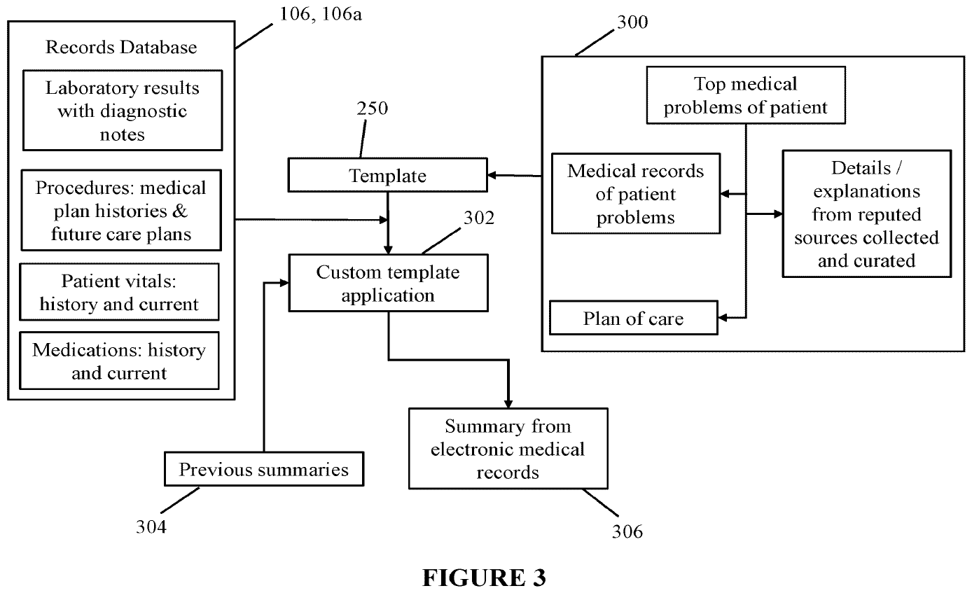

FIG. 3 illustrates an exemplary demonstration of at least portions of the template 250 being generated and customized to derive a summary of electrical medical records, as indicated by block 306, for consideration for inclusion in the communication content. As shown in FIG. 3, the records database 106a can include information from a variety of different sources, and or a variety of different types of information. For example, according to certain embodiments, the records database 106a can include information obtained from laboratory results, such as, for example, results of measurements and or analysis of specimens or samples obtained from the patient, including, but not limited to, analysis of a blood sample from the patient. Further, in at least certain instances, such laboratory results can also include diagnostic notes, such as, for example, notes, comments, and/or analysis provided by one or more members of the first group, including, for example, laboratory technicians, among others. Additionally, or alternatively, the records database 106a can include historical and/or current information relating to the patient, such as, for example, with respect to past, current, or future medical procedures that the patient has/will undergone/undergo, with respect to clinical data, such as, for example, vital signs of the patient, and/or with respect to medications or associated medication prescriptions for, or taken by, the patient.

Certain criteria (as generally indicated by block 300 in FIG. 3) for at least portions of the template 250 that is associated with type of information that may be stored or retrieved by/from the records database 106a. For example, as indicated by block 300, portions of the template 250 pertaining to the type of information that can received/retrieved from the records database 106a can pertain to categories such as top medical problems, medical records, plans of care, and/or details or explanations from reputed sources collected and curated. Moreover, each of these types of criteria can, for example, further identify at least types of information that may be relevant for at least purposes of customizing at least portions of the template 250 for the patient. Moreover, such criteria can, along with information provided by the records database 106a, can be used to determine which information is, or is not, to be populated in the template 250 for that particular patient, as discussed below.

Accordingly, in the exemplary embodiment illustrated in FIG. 3, the identified criteria (block 300) can be utilized to generate or identify at least potentially pertinent portions of the template 250. As indicated by the arrows in FIG. 3, information provided by the records database 106a can be applied to at least portions of the template 250 that was generated using at least criteria such as that shown, for example, in block 300, to generate a custom template, as shown in block 302. For example, information provided from the records database 106a and/or the criteria provided at block 300 can be utilized at block 302 in the manual and/or automatic identification of which information that is, or is not, to be populated for at least a portion of the template that is to be at least considered for conveying patent information to the surrogate in the communication content.

Further, the application of information from the records database 106a to custom template can be used to generate, at block 306, a summary from electronic medical records, as generally indicated by block 304. Additionally, in at least certain instances, the generation of the summary from electronic medical records can include, at block 302, receipt of information in the previously generated summaries of electronic medical records (block 304), if any. Such previous summaries can be utilized for a variety of purposes. For example, according to certain embodiments, the information from such summaries from block 304 can be utilized at block 302 with the information in the current custom template, including from the records database 106a, to further assist in identifying information that is to be included in the custom template. Additionally, or alternatively, information provided by the previous summaries from block 304 can be used with information that is to be applied to the template from the records database 106a to identify trends and or changes in the status and or condition of the patient. Additionally, or alternatively, information provided by the previous summaries from block 304 can be compared with information in the current custom template at block 302 in connection with evaluating the accuracy of either current, or prior, information from the records database 106a. Thus, for example, according to certain embodiments, the previous summaries can, among other functions or purposes, be utilized at least in connection with performing verification of information being used at block 302.

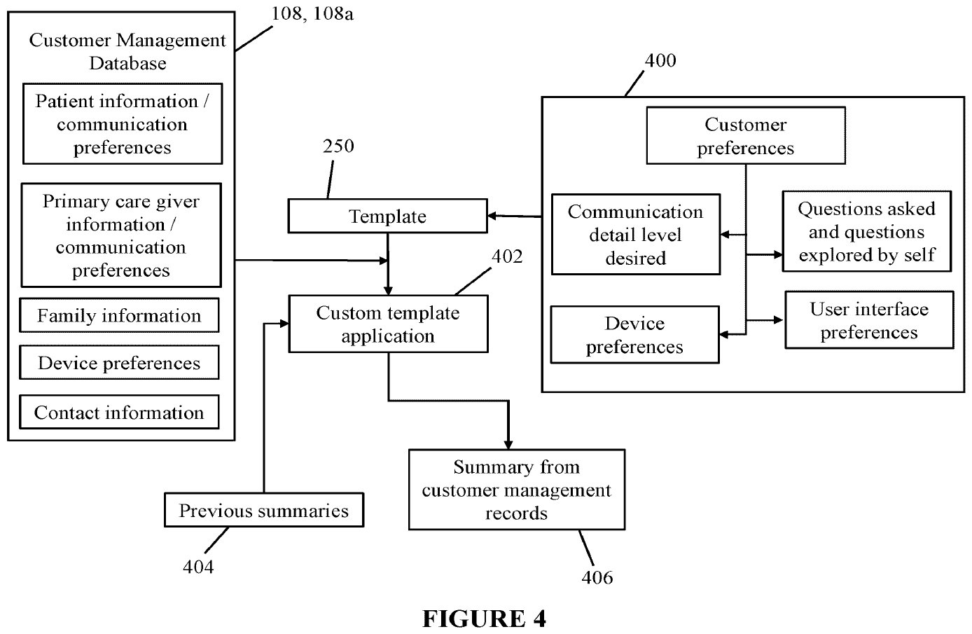

FIG. 4 illustrates an exemplary demonstration of at least portions of the template 250 being generated and customized to derive a summary of customer relationship management records, as indicated by block 406, for consideration for inclusion in the communication content that is to be communicated to the one or more surrogates of a second group. As shown in FIG. 4, the customer management database 108a can include a variety of different types of information. For example, according to certain embodiments, the customer management database 108a can include preference information obtained from a patient, also be referred to as a customer, including patient identification and communication preference information. For example, the patient can provide contact information, including numbers and/or addresses for telephonic or electronic communications, as well as addresses for mail delivery, including, but not limited to, electronic mail (email) addresses. Such communication preferences can also, for example, pertain to the manner in which the patient is to be contacted, including, for example, whether there is a preference for communications via telephone, mail, or electronic communications, and whether the preferences for the manner in which communications are to be made changes for different times of the day.

The customer management database 108a can further include information regarding a primary caregiver for the patient such as, for example, identification and contact information. Such information regarding the primary caregiver can further include, for example, communication preferences (e.g., mail, telephone, and/or electronic communications, among other), and/or the time(s) of day to contact the primary caregiver, among other information. The customer management database 108a can further include information regarding the family of the patient, such as, for example, and identification and associated contact information of one or more family members that is/are to act as a surrogate in the event the patient is not capable of making decisions regarding the healthcare of the patient. Such family information can also indicate preferences with respect to the type or level of patient information a surrogate is, or is not, to have access. Further, the customer management database 108a can include information regarding the particular recipient device 118, including, for example, particular cellular or mobile phones, that are to receive communications from the healthcare system 100.

Certain criteria (as generally indicated by block 400 in FIG. 4) for at least the portion of the template 250 that is associated with the type information that may be provided by the customer management database 108a can correspond to categories regarding customer preferences, communication detail level desired, device preferences, questions asked and or explored by the patient, and/or user interface preferences. For example, the communication detail level desired category for the template 250 can correspond to the type of information that may or may not be provided in a recorded message or electronic message for the patient. Additionally, the communication detail level category can correspond to the extent, or degree, information can be communicated to at least certain comma if not all comma designated surrogates. The device preferences category and/or user interface preferences can correspond to the particular devices of the patient, and manners available to receive communications on such devices (e.g., telephonic, text, email), of the patient and/or surrogates to which information may or may not be communicated. Moreover, each of these types of criteria can, for example, further identify at least types of information that may be relevant for at least purposes of customizing at least portions of the template 250 using to the type of information stored or retrievable from the customer management database 108a.

Accordingly, in the exemplary embodiment illustrated in FIG. 4, the identified criteria (block 400) can be utilized to manually and/or automatically generate at least portions of the template 250. Thus, as indicated by the arrows in FIG. 4, information provided by the customer management database 108a can be applied to at least portions of the template 250 that were generated using at least criteria such as that shown, for example, in block 400 in connection with generating a custom template, as indicated in block 402. Additionally, the custom template generated at block 402, and/or the information provided by the custom management database 108a, can be used to populate the associated portion(s) of the template. Moreover, the application of at least information from the custom management database 108a to the associated portion(s) of the template can be used to for the generation of a summary from customer management records (block 406).

In at least certain instances, the application of information from the customer management database 108a to custom template can be used to generate the summary from electronic medical records, can also involve consideration of previously generated summaries (block 404), such as, for example, previously summary from customer management records. Such previous summaries can be utilized for a variety of purposes. For example, according to certain embodiments, the information from such summaries from block 404 can be utilized at block 402 with the information in the current custom template, including from the customer management database 108a, to further assist in identifying information that is to be included in the custom template. Additionally, or alternatively, information provided by the previous summaries from block 404 can be used with information that is to be applied to the template from the customer management database 108a to identify changes, if any, in the associated information. Additionally, or alternatively, information provided by the previous summaries from block 404 can be compared with information in the current custom template at block 402 in connection with evaluating the accuracy of either current, or prior, information from the database 106a. Thus, for example, according to certain embodiments, the previous summaries can, among other functions or purposes, be utilized at least in connection with performing verification of information being used at block 402.

FIG. 5 illustrates an exemplary method 500 of gathering communication content for review by one or members of the first group and that can be utilized for generating communication content for the recipient device 118. The method 500 is described below in the context of being carried out by the illustrated exemplary healthcare communication system 100. However, it should be appreciated that method 500 can likewise be carried out by any of the other described implementations, as well as variations thereof. Further, the method 500 corresponds to, or is otherwise associated with, performance of the blocks described below in the illustrative sequence of FIG. 5. It should be appreciated, however, that the method 500 can be performed in one or more sequences different from the illustrative sequence. Additionally, one or more of the blocks mentioned below may not be performed, and the method 500 can include steps or processes other than those discussed below.

As demonstrated in at least FIG. 5, at step 502, the communication content from the summary from the inter-disciplinary team, as discussed, for example, at block 210 of FIG. 2, the summary from electronic records, as discussed with respect to block 306 of FIG. 3, and the summary from the customer management records, as discussed with respect to block 406 of FIG. 4, among other information, can be aggregated by the communication content aggregator and curator system 110, including by the aggregator and curator system 114. Thus, for example, the derived summary information discussed with respect to at least blocks 210, 306, and 406b in FIGS. 2, 3, and 4, respectively, can be aggregated at block 502.

According to certain embodiments, the derived summary information discussed with respect to at least blocks 210, 306, and 406b may already be formatted in a manner in which the summary information in blocks 210, 306, and 406b each provide, for form, different portions of the template. Thus, for example, the summary information provided by blocks 210, 306, 406 can, according to certain embodiments, be different portions of and overall template used by at least the communication content and aggregator system 110. Thus, in such situations, the summaries aggregated at block 502 can, at block 504, be compiled to provide an overall template for the communication content that is to be considered for transmission or communication to the surrogate Alternatively, the summary information from one or more of blocks 210, 306, 406 can be information that was collected for the summaries based on the type of information that was, or was anticipated to be, used for a template, and such information can then be used in connection with generating a template at block 504. For example, the information in the aggregate summaries can be utilized at block 504 for a manual and/or automatic selection of information for the template, and the associated population of that template. Such population can therefore provide various information regarding the condition, treatment, and/or prognosis of the patient, and can further include information provided by the aggregated summaries from block 502.

Regardless of whether the aggregated summaries from block 502 are, or are not, in a form that corresponds to at least portions of a template, according to certain embodiments, the template can, at block 504, be generated and/or refined so as to include fields or categories that correspond to the different types of information that could be provided from the input sources 102, 104, 106, 108, including, for example categories of information, as discussed above with respect to at least FIGS. 2-4. Thus, according to certain embodiments, block 504 can include a determination of which fields are to be populated for the template that will undergo supervisory review by a member of the first group. Further, the template generated for at least supervisory review, if not for communication to the surrogate, can include, but is not limited to, for example, reporting information, observations, instructions, and/or explanations, among information, that can be relevant to the particular patient's condition, situation, treatment, and/or prognosis, among other pertinent information. Moreover, at block 504 the information that was aggregated at block 502 can be refined with respect to the particular information that may be eventually communicated to the member(s) of the second group. Such refinement can be utilized, for example, to control the amount of information that may be delivered to the second group, including, for example, the surrogate.

Thus, the curation template reference by block 504 in FIG. 5 can, for example, be a compilation of the custom template application discussed above with respect to at least the customized templates discussed above with respect to at least blocks 208, 302, 402. Thus, the curation template at block 504 can be based on, or utilize criteria similar to the criterial discussed above with respect to at least blocks 206, 300, 400. Accordingly, as indicated by block 506 in FIG. 5, the criteria utilized in connection with at least the template referenced by block 504 can include, or be supplemented by, for example, criteria such as, for example, top or prevalent medical problems, relevant or pertinent information needed for decision making by the surrogate, the level of information and communication preferences of the surrogate and/or patient, anticipated questions from the surrogate/patient, anticipated frequently asked questions (FAQs), and/or previously provided answers or responses, among other criteria.

Accordingly, at block 506, the template generated or compiled at block 504 can be further customized or tailored to the specific issues, concerns, and/or history of the patient at issue. For example, as indicated in FIG. 5, the template can be populated, and/or supplemented, to address relevant information that a family may need relating to the patient. Additionally, based on the type of information aggregated at bock 504, and/or prior experiences with the second group and/or prior patients in similar situations, the template can also be tailored to include answers or guidance to anticipated questions. Additionally, block 506 can also include information regarding the communication form selected by the second group, such as, for example, whether the communication is to be provided via a letter or text message, among other forms of communication that may impact the format of the template. Accordingly, the template curated at block 504 can provide communication content having information at least relating to an associated patient, and/or from the first group, that can provided the basis for, or be, the communication content that is at least proposed for communicating to the surrogate, including, for example, be proposed for communicating to the recipient device 118 of one or more surrogates of the second group

At block 508, the proposed communication content can undergo review for supervisory approval. For example, at block 508, a member of the first group who is to review and/or approve the communication content can have the ability to remove, edit, and/or supplement the communication content. Such editing and/or supervision can, according to certain embodiments, be performed via an associated care provider device 112 and/or via use of the user interface 119 of the curation system 116.

Following approval at block 508, and any associated modification of the communication content, at block 510 the approved content can be presented for consumption by the second group on their recipient device 118 or contextual situation. Thus, for example, at block 510, be communicated content can be communicated from the curation system 116 of the communication content aggregator and curator system 110 so as to be provided, or otherwise available from, at least the recipient device 118. Additionally, or alternatively, following supervisory approval, the authorized version of the communication content can be accessible to the selected member(s) of the second group, such as, for example, via at least the display 131 or other device of the user interface 129 of the curation system 116.

Additionally, or optionally, according to certain embodiments, the method 500 can also include the communication content aggregator and curator system 110 generating one or more signals that facilities the communication of reminders to the surrogate, and, moreover, to the recipient device 118. Such reminder communications can be intended to facilitate a response and/or action by the surrogate or other member(s) of the second group.

In view of the foregoing, the subject disclosure can provide an efficient and effective system and process that can improve the ease in communication between members of the first and second groups, such as, for example, ICU families, physicians and other service providers in ICU, among other groups of employees, teams, agents, and individuals or collections of individuals. Additionally, the disclosure herein is applicable to a variety of different groups, including, for example, different patient groups and their family and friends. The enhanced communication content provided by the subject disclosure can produce meaningful engagement with the families and friends of patients and ease some of the hospitalization associated stress. The engagement can also ease the decision making that is imposed on the family members and/or friends of patients. While the above includes specific reference to ICU patients and family members, among other patients, the subject disclosure is applicable to a wide variety of patients, including inpatients and outpatients, and can also be applicable to other industries or service and business segments. Additionally, the communications discussed herein can relate to observations and/or treatments not only when a patient is in a hospital setting, but also during various stages of recovery, rehabilitation, and/or in-home settings, as well as other subsequent time periods during or following a recovery process.

Additionally, as exemplified by the above-discussed disclosure, embodiments of the subject disclosure can provide a comprehensive, easy-to-use communication platform for patients, family/friends of patients, and surrogate decision makers. Benefits of the subject disclosure can also include a savings of time for professional care providers due to auto-development of communication materials, as well as for nurses and staff through reduction in repeated communication of duplicative information to patient families and friends. The subject disclosure also can improve situation awareness for at least members of the second group, which can lead to better and less stressful decision making for families.

As discussed above, embodiments disclosed herein can provide a custom and relevant summary of information from the hospital or provider healthcare system for patient, surrogate and family members/friends to consume and be situationally aware. Additionally, the disclosed embodiments can provide a customer-situation specific generation of relevant summary of information through novel aggregation of information from input sources 102, 104, 106, 108 that can include rounds information, as well as from information provided by electronic medical records information and customer relationship management information. Additionally, the disclosed embodiments can accommodate supervisory input from a care provider, including one or more members and/or a team of the care provider, to an automated communication for better communication of medical information and also maintenance of proper meaningful dialogue.

While the invention has been described in connection with what is presently considered to be the most practical and preferred embodiment, it is to be understood that the invention is not to be limited to the disclosed embodiment(s), but on the contrary, is intended to cover various modifications and equivalent arrangements included within the spirit and scope of the appended claims, which scope is to be accorded the broadest interpretation so as to encompass all such modifications and equivalent structures as permitted under the law. Furthermore it should be understood that while the use of the word preferable, preferably, or preferred in the description above indicates that feature so described may be more desirable, it nonetheless may not be necessary and any embodiment lacking the same may be contemplated as within the scope of the invention, that scope being defined by the claims that follow. In reading the claims it is intended that when words such as “a,” “an,” “at least one” and “at least a portion” are used, there is no intention to limit the claim to only one item unless specifically stated to the contrary in the claim. Further, when the language “at least a portion” and/or “a portion” is used the item may include a portion and/or the entire item unless specifically stated to the contrary.

1. A healthcare system comprising:
a first database having an observation data pertaining to a patient;
a second database having a historical medical data for the patient;
a third database having a customer preference data for the patient;
a communication content aggregator and curator system having at least one processor and a memory coupled with the at least one processor, the memory including instructions that when executed by the at least one processor cause the at least one processor to:
aggregate data at least obtained from the observation data, the historical medical data, and the customer preference data;
curate the aggregated data to provide a curated template, the curated template being populated based at least in part on information provided by the aggregated data;
generate a communication content comprising information from the curated template; and
generate, in response to a receipt of a signal that indicates a supervisory approval of the communication content by one or more members of a first group, at least a signal that provides and/or indicates access to the communication content to at least one member of a second group.

2. The healthcare system of claim 1, wherein the first group comprises at least one or more medical professionals of a healthcare provider that provides medical services for the patient, and the at least one member of the second group comprises a surrogate that is not the patient.

3. The healthcare system of claim 1, wherein the memory includes instructions that when executed by the at least one processor further cause the at least one processor to receive the signal that indicates an occurrence of the supervisory approval of the communication content.

4. The healthcare system of claim 3, wherein the signal that provides and/or indicates access to the communication content communicates the communication content to a recipient device of the at least one member of the second group in a form of a written communication.

5. The healthcare system of claim 1, wherein the curated template is populated based at least in part on an indication from the aggregated data of a type of medical problem of the patient.

6. The healthcare system of claim 5, wherein the communication content includes responds to prior questions from the at least one member of the second group.

7. The healthcare system of claim 1, wherein the memory includes instructions that when executed by the at least one processor further cause the at least one processor to adjust the communication content in response to information included in one or more prior communication contents that had previously been accessible to the at least one member of the second group.

8. The healthcare system of claim 1, wherein the memory includes instructions that when executed by the at least one processor further cause the at least one processor to generate a signal to request a response from the at least one member of the second group regarding a condition of the patient.

9. The healthcare system of claim 8, wherein the memory includes instructions that when executed by the at least one processor further cause the at least one processor to derive information from one or more responses provided by the at least one member of the second group regarding the condition of the patient, and wherein the aggregated data includes at least a portion of the one or more responses provided by the at least one member of the second group.

10. The healthcare system of claim 8, wherein the memory includes instructions that when executed by the at least one processor further cause the at least one processor to generate a signal for a recipient device of the at least one member of the second group that reminds the at least one member of the second group of the request for the response regarding the condition of the patient.

11. A method comprising:
accessing observation data pertaining to a patient;
accessing historical medical data for the patient;
accessing customer preference data for the patient;
aggregating the observation data, the historical medical data, and the customer preference data to obtain a set of aggregated data;
curating the set of aggregated data to provide a curated template;
generating a communication content comprising information from the curated template; and
generating, in response to a receipt of a signal that indicates a supervisory approval of the communication content by one or more members of a first group, at least a signal that provides and/or indicates access to the communication content to at least one member of a second group.

12. The method of claim 11, further comprising populating the curated template based at least in part on information provided by the set of aggregated data, and wherein the first group comprises at least one or more medical professionals of a healthcare provider that provides medical services for the patient.

13. The method of claim 12, further comprising identifying, using at least the observational data, a type of medical problem of the patient, and wherein populating the curated template is based at least in part on the identified type of medical problem.

14. The method of claim 11, further comprising receiving a signal that indicates a granting of the supervisory approval.

15. The method of claim 11, wherein generating the signal that provides and/or indicates access to the communication content to the at least one member of the second group comprises providing access to the communication content in a written format.

16. The method of claim 11, further comprising deriving information from one or more communications from the at least one member of the second group, and wherein the set of aggregated data includes the information derived from the one or more communications from the at least one member of the second group.

17. The method of claim 11, further comprising adjusting the communication content based on information provided by one or more prior communication contents that had previously been accessible to the at least one member of the second group.

18. The method of claim 11, further comprising generating a signal to request a response from the at least one member of the second group regarding a condition of the patient.

19. The method of claim 18, further comprising deriving information from one or more responses provided by the at least one member of the second group regarding the condition of the patient, and wherein the set of aggregated data includes at least a portion of the one or more responses provided by the at least one member of the second group.

20. The method of claim 18, further comprising generating a signal for a recipient device of the at least one member of the second group that reminds the at least one member of the second group of the request for the response regarding the condition of the patient.
